(19) United States
(12) Patent Application Publication
(10) Pub. No.: US 2014/0155294 A1
(43) Pub. Date: Jun. 5, 2014

(54) CELL CHIP, CELL SLICE SAMPLE AND MANUFACTURING METHOD THEREOF

(71) Applicant: SAMSUNG ELECTRO-MECHANICS CO., LTD., Suwon (KR)

(72) Inventors: Dong Woo LEE, Suwon (KR); Bo Sung KU, Suwon (KR)

(73) Assignee: SAMSUNG ELECTRO-MECHANICS CO., LTD., Suwon (KR)

(21) Appl. No.: 13/770,910

(22) Filed: Feb. 19, 2013

(30) Foreign Application Priority Data
Dec. 5, 2012 (KR) 10-2012-0140579

(51) Int. Cl. G01N 1/36; C12N 11/00

(52) U.S. Cl. CPC G01N 1/36; C12N 11/00
USPC 506/14; 435/40.52; 506/26

(57) ABSTRACT

There is provided a cell chip, a cell slice sample, and a manufacturing method thereof. The cell chip includes a plate member; biomaterials attached to one surface of the plate member; and a solidifiable material formed on one surface of the plate member to form a layer including the biomaterials.

### CROSS-REFERENCE TO RELATED APPLICATIONS

This application claims the priority of Korean Patent Application No. 10-2012-0140579 filed on Dec. 5, 2012, in the Korean Intellectual Property Office, the disclosure of which is incorporated herein by reference.

### BACKGROUND OF THE INVENTION

1. Field of the Invention

The present invention relates to a cell chip, a cell slice sample, and a manufacturing method thereof, and more particularly, to a cell chip and a cell slice sample allowing for easiness of inspection and analysis, and a manufacturing method thereof.

2. Description of the Related Art

The demand for biomedical devices and biotechnology for rapidly diagnosing various human diseases of has recently increased. Therefore, the use of cell chips allowing for experiments to be performed on various biomaterials and drugs simultaneously has increased. Since cell chips include a plurality of biomaterials or drugs, reaction results with respect to the plurality of biomaterials or drugs may be observed after performing a single experiment.

However, it is complicated to manufacture the cell chip and it takes a great deal of time and labor to manufacture the cell chip. As an example of the Related Art, there is provided Patent Document 1 relating to cell chips. However, a technology disclosed in Patent Document 1 has a disadvantage in that a large number of processes should be performed in order to fix a biopolymer to a substrate 1.

Meanwhile, as another experimental method using a biomaterial, Patent Document 2 discloses a method of using a fixed material. Patent Document 2 relates to a technology of accommodating tissue 8 in a hole 4 of a paraffin block 2, slicing the tissue, and using the slice. However, since the technology disclosed in Patent Document 2 uses a scheme of inserting the tissue into the hole 4, it has a disadvantage that a significantly large amount of tissue is required (it may be difficult to secure a large amount of biological tissue, since the biological tissue is highly expensive). In addition, in the case of pre-treating (culturing or medicating) the tissue 8, an inconvenient operation of pre-treating each sample of biological tissue using a separate device and moving this sample in order to slice the sample is required.

### RELATED ART DOCUMENT

(Patent Document 1) JP2003-021635 A
(Patent Document 2) JP2011-013052 A

### SUMMARY OF THE INVENTION

An aspect of the present invention provides a cell chip and a cell slice sample capable of decreasing time and costs required for an experiment using a biomaterial by performing a process of pre-treating a biological tissue and a process of slicing the biological tissue on the same substrate, and a manufacturing method thereof.

According to an aspect of the present invention, there is provided a cell chip including: a plate member; biomaterials attached to one surface of the plate member; and a solidifiable material formed on one surface of the plate member to form a layer including the biomaterials.

The plate member may include a hydrophilic region to which the biomaterials are attached and a hydrophobic region blocking the biomaterials from being attached thereto.

The hydrophobic region may be formed to enclose the hydrophilic region.

The plate member may include protrusions to which the biomaterials are attached.

End surfaces of the protrusions may be coated with a hydrophilic material so that the biomaterials may be easily attached thereto.

The solidifiable material may include a paraffin component.

The biomaterials may be sampled from at least one kind of sample.

According to another aspect of the present invention, there is provided a cell slice sample including: a slice formed of a solidifiable material; and at least one kind of biomaterials formed on the slice.

The slice may be formed of a paraffin component.

The biomaterials may be provided at predetermined intervals in first and second directions of the slice.

According to another aspect of the present invention, there is provided a method of manufacturing a cell chip, including: attaching biomaterials to one surface of a plate member; and forming a solidifiable layer on one surface of the plate member, the solidifiable layer accommodating the biomaterials therein and being formed of a solidifiable material.

The method may further include: applying a hydrophobic material to the plate member; and partially applying a hydrophilic material to the hydrophobic material.

According to another aspect of the present invention, there is provided a method of manufacturing a cell chip, including: attaching biomaterials to protrusions formed on one surface of a plate member; and forming a solidifiable layer on one surface of the plate member, the solidifiable layer accommodating the biomaterials therein and being formed of a solidifiable material.

The method may further include: applying a hydrophobic material to the plate member; and partially applying a hydrophilic material to the hydrophobic material.

The attaching of the biomaterials may include immersing the protrusions of the plate member in grooves in which the biomaterials are accommodated.

According to another aspect of the present invention, there is provided a method of manufacturing a cell slice sample, including: attaching biomaterials to one surface of a plate member; forming a solidifiable layer on one surface of the plate member, the solidifiable layer accommodating the biomaterials therein and being formed of a solidifiable material; and cutting the solidifiable layer into a slice.

The method may further include: applying a hydrophobic material to the plate member; and partially applying a hydrophilic material to the hydrophobic material.

According to another aspect of the present invention, there is provided a method of manufacturing a cell slice sample, including: attaching biomaterials to protrusions formed on one surface of a plate member; forming a solidifiable layer on one surface of the plate member, the solidifiable layer accommodating the biomaterials therein and being formed of a solidifiable material; and cutting the solidifiable layer into a slice.

The method may further include applying a hydrophobic material to the protrusions.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view of a cell chip according to an embodiment of the present invention;

FIG. 2 is a cross-sectional view of the cell chip taken along line A-A of FIG. 1;

FIG. 3 is a cross-sectional view taken along line A-A illustrating another form of a plate member shown in FIG. 2;

FIG. 4 is a cross-sectional view taken along line A-A illustrating another form of the plate member shown in FIG. 2;

FIG. 5 is a cross-sectional view of a cell chip according to another embodiment of the present invention taken along line A-A;

FIG. 6 is a cross-sectional view taken along line A-A illustrating another form of a plate member shown in FIG. 5;

FIG. 7 is a view illustrating a method of manufacturing a cell chip according to an embodiment of the present invention;

FIG. 8 is a view illustrating a method of manufacturing a cell chip according to another embodiment of the present invention;

FIG. 9 is a perspective view of a cell slice sample according to an embodiment of the present invention;

FIG. 10 is a view illustrating a method of manufacturing a cell slice sample according to an embodiment of the present invention; and

FIG. 11 is a view illustrating a method of manufacturing a cell slice sample according to another embodiment of the present invention.

### DETAILED DESCRIPTION OF THE EMBODIMENTS

Hereinafter, embodiments of the present invention will be described in detail with reference to the accompanying drawings.

The invention may, however, be embodied in many different forms and should not be construed as being limited to the embodiments set forth herein. Rather, these embodiments are provided so that this disclosure will be thorough and complete, and will fully convey the scope of the invention to those skilled in the art.

In the drawings, the shapes and dimensions of components may be exaggerated for clarity, and the same reference numerals will be used throughout to designate the same or like components.

For reference, a plate member described in the present specification refers to a member used for performing an experiment on a biomaterial, and a material for the plate member is not particularly limited. Therefore, a plate member to be described below may be formed of silicon, glass, metal, or polymer. Here, a kind of polymer is not particularly limited, but may be, for example, polymethylmethacrylate (PMMA), polycarbonate (PC), polystyrene (PS), polypropylene, a cyclic olefin copolymer, polynorbonene, a styrene-butadiene copolymer (SBC), or acrylonitrile butadiene styrene.

In addition, a method of manufacturing the plate member as described above is not particularly limited. For example, the plate member may be manufactured by a photo-resist process, an etching process, an injection molding process, or the like.

In addition, a biomaterial mentioned in the present specification refers to various materials. For example, the biomaterial may be nucleic acid arrangement such as ribonucleic acid (RNA), deoxyribonucleic acid (DNA), or the like, peptide, protein, lipid, organic or inorganic chemical molecules, virus particles, prokaryotic cells, organelles, or the like. In addition, the biomaterial is not limited to human cells, but may be used in the sense of including cells of various animals or plants.

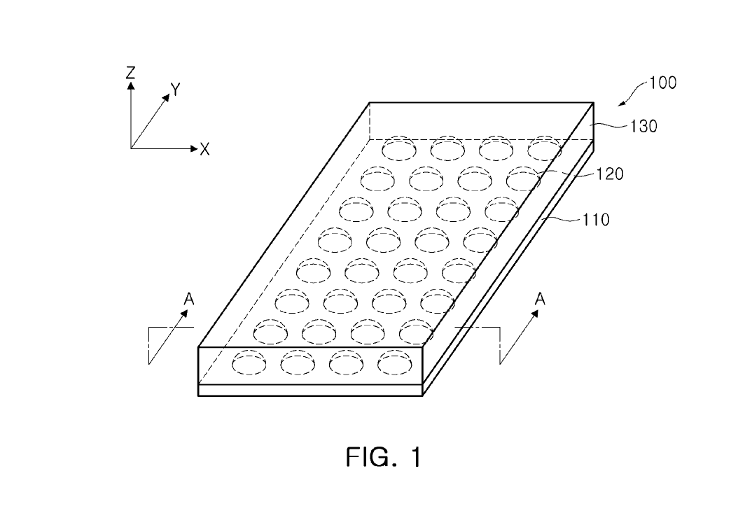

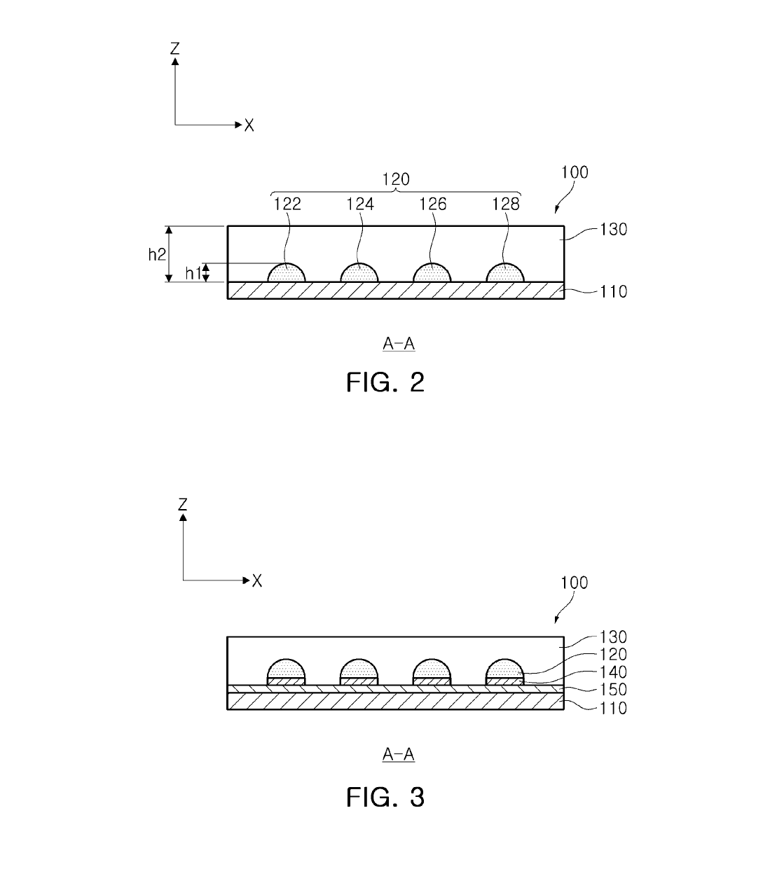

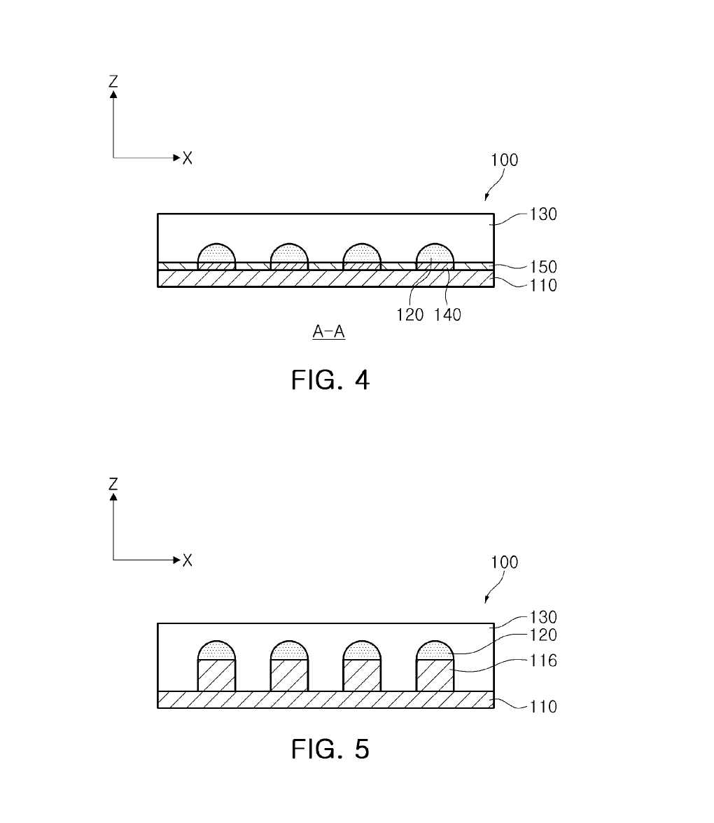

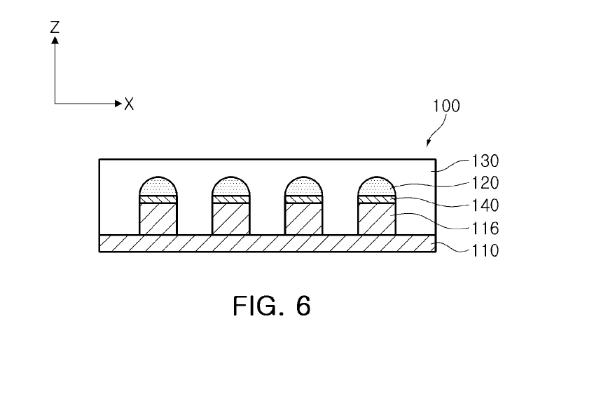

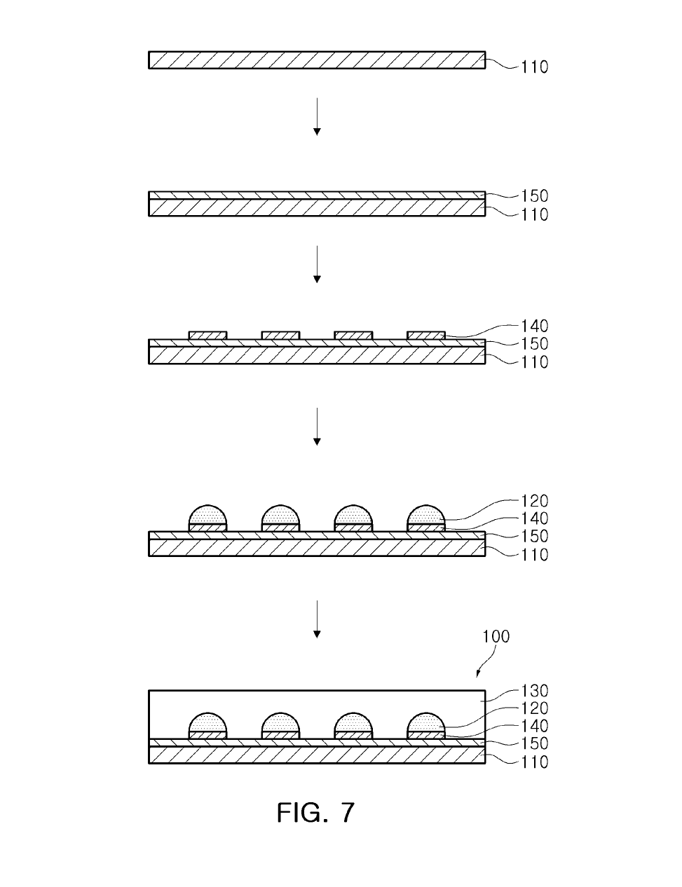

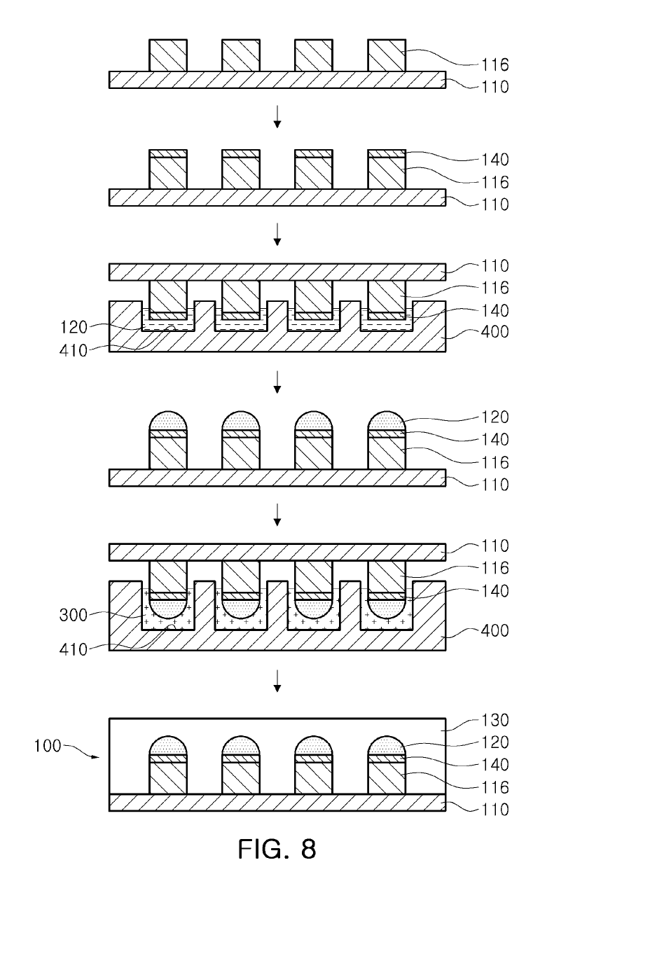

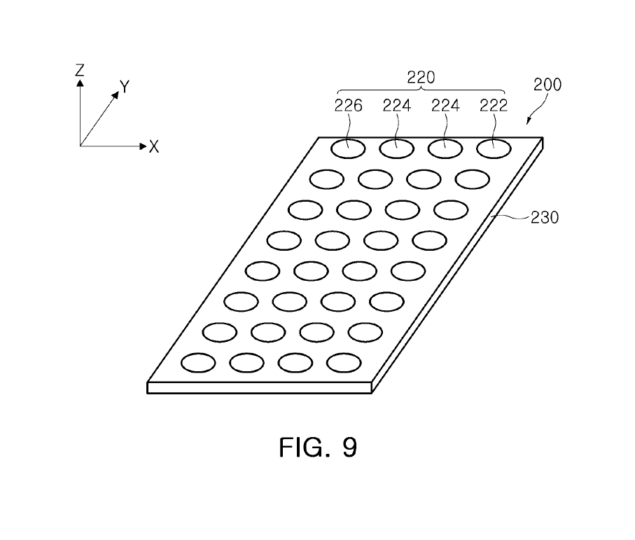

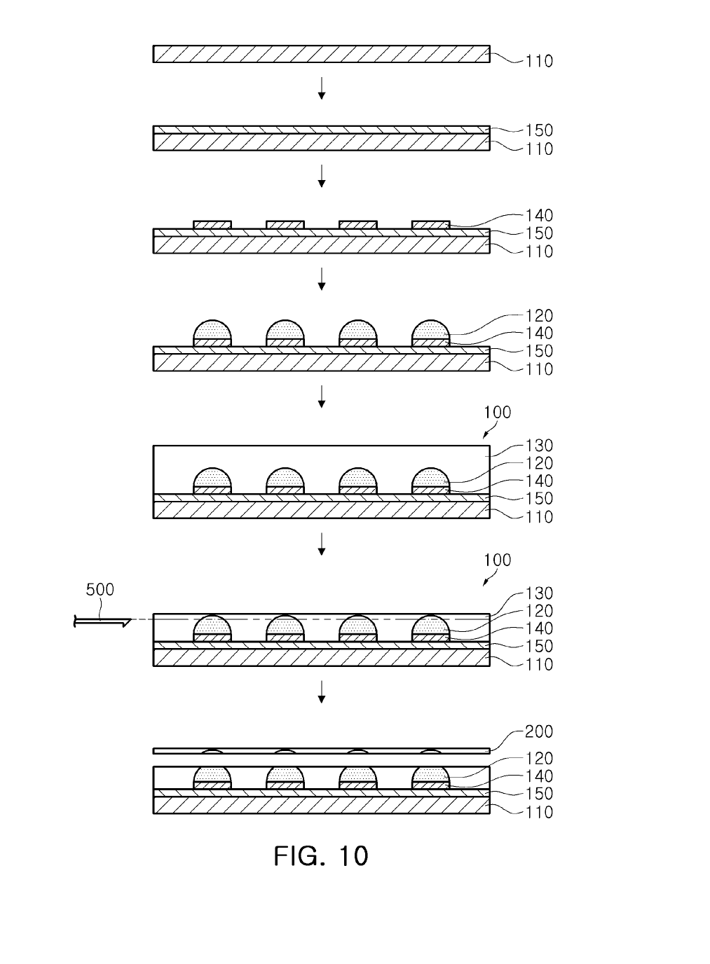

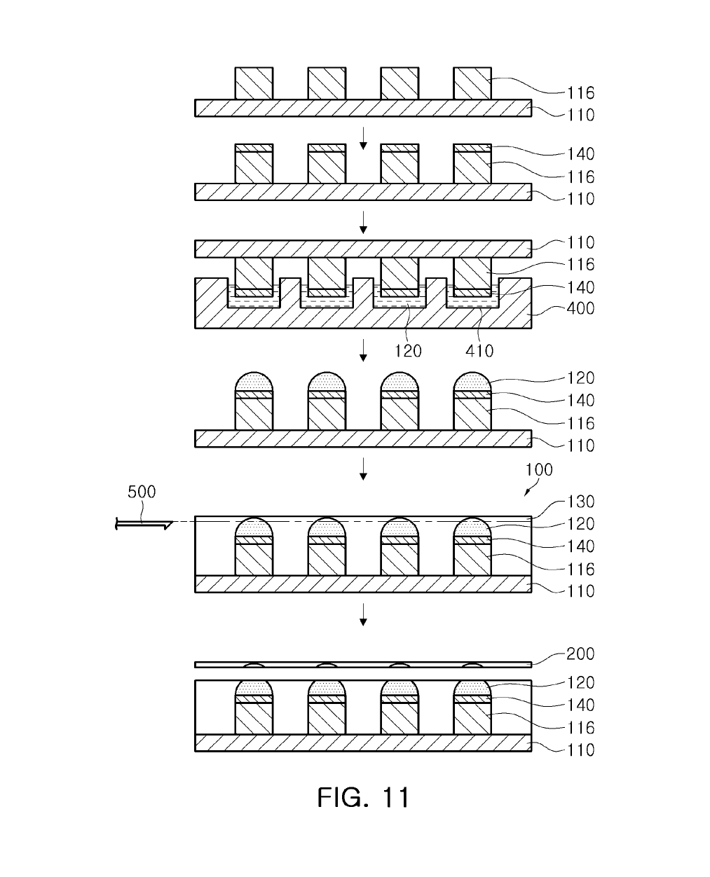

FIG. 1 is a perspective view of a cell chip according to an embodiment of the present invention; FIG. 2 is a cross-sectional view of the cell chip taken along line A-A of FIG. 1; FIG. 3 is a cross-sectional view taken along line A-A illustrating another form of a plate member shown in FIG. 2; FIG. 4 is a cross-sectional view taken along line A-A illustrating another form of a plate member shown in FIG. 2; FIG. 5 is a cross-sectional view of a cell chip according to another embodiment of the present invention taken along line A-A; FIG. 6 is a cross-sectional view taken along line A-A illustrating another form of a plate member shown in FIG. 5; FIG. 7 is a view illustrating a method of manufacturing a cell chip according to an embodiment of the present invention; FIG. 8 is a view illustrating a method of manufacturing a cell chip according to another embodiment of the present invention; FIG. 9 is a perspective view of a cell slice sample according to an embodiment of the present invention; FIG. 10 is a view illustrating a method of manufacturing a cell slice sample according to an embodiment of the present invention; and FIG. 11 is a view illustrating a method of manufacturing a cell slice sample according to another embodiment of the present invention.

A cell chip according to an embodiment of the present invention will be described with reference to FIGS. 1 through 4.

A cell chip 100 according to the embodiment of the present invention may include a plate member 110, biomaterials 120, and a solidifiable material 130.

The plate member 110 may have a rectangular shape. More specifically, the plate member 110 may have a rectangular shape in which it is extended lengthwise in one direction (a Y axis direction, based on FIG. 1). However, the plate member 110 is not limited to having the rectangular shape, but may have other shapes as needed.

The plate member 110 may be formed of silicon, glass, metal, or a polymer. More specifically, the plate member 110 may be made of a material that may be easily molded. In addition, the plate member 110 may be formed of a material that does not biologically and chemically react to the biomaterials 120.

The plate member 110 may have a size appropriate be observed by a microscope and be optically analyzed. For example, the size of the plate member 110 may be the same as or smaller than a glass slide. The plate member 110 having the small size as described above may be easily carried and stored.

The biomaterials 120 may be provided on the plate member 110. More specifically, the biomaterials 120 may be provided at predetermined intervals on one surface of the plate member 110 by a fine discharging device (for example, a pipette). Here, the biomaterials 120 provided on the plate member 110 may have a small amount, on the level of several tens of μl.

The biomaterials may be maintained in a hemispherical shape or a similar shape thereto as shown in FIG. 2 by surface tension. That is, the biomaterials 120 may be present at a predetermined height h1 from one surface of the plate member 110.

The biomaterial 120 may be a partial tissue of an animal group including a person. More specifically, the biomaterial 120 may be organ tissue or skin tissue of a person or organ tissue or skin tissue of an animal. However, the biomaterial 120 is not limited to human or animal tissue as described above. For example, the biomaterial may be used in the sense of including tissue of a plant and a microorganism.

Meanwhile, the biomaterials 120 may be at least one kind of tissue. For example, a first row among the biomaterials 120 provided on the plate member 110 may be a first tissue 122, a second row thereamong may be a second tissue 124, a third row thereamong may be a third tissue 126, and a fourth row thereamong may be a fourth tissue 128. When the biomaterials 120 are arranged as described above, there is an advantage that reactions of several tissues to a single type of drug or reagent may be simultaneously observed. In addition, the arrangement of the biomaterials 120 as described above may enable observation of an interaction between the first to fourth tissues 122 to 128 with respect to a single type of drug or reagent.

The solidifiable material 130 may be provided on one surface of the plate member 110. More specifically, the solidifiable material 130 may form a single layer having a predetermined height on one surface of the plate member 110. Here, the layer of the solidifiable material 130 may accommodate the biomaterials 120 therein and solidify the biomaterials 120. To this end, the solidifiable material 130 may be formed of a paraffin component or a mixture including the paraffin component.

The layer (hereinafter, referred to as a solidifiable layer and denoted by the same reference numeral as that of the solidifiable material 130) of the solidifiable material 130 may be formed to have a predetermined height h2 from one surface of the plate member 110. Here, the height h2 of the solidifiable layer 130 may be the same as or larger than the height h1 of the biomaterial 120. The former may be advantageous for manufacturing a cell slice sample 200 using the cell chip 100, and the latter may be advantageous for protecting and solidifying the biomaterials 120 through the solidifiable layer 130.

In the cell chip 100 configured as described above, since the biomaterials 120 provided on the plate member 110 are protected by the solidifiable layer 130, modification and damage of the biomaterials 120 due to external impact or external contact may be prevented. In addition, since the biomaterials 120 are solidified by the solidifiable layer 130, the cell chip 100 may be advantageous for storing the biomaterials 120 for an extended period of time. Further, in the cell chip 100, since the biomaterials 120 may be solidified integrally with the solidifiable layer 130, the biomaterials 120 may be manufactured to be a plurality of slices through a process of cutting the solidifiable layer 130. As a result, different types of drug experiments may be performed even by a single cell chip 100.

Next, another form of the plate member 110 will be described with reference to FIGS. 3 and 4.

The plate member 110 may be divided into a hydrophilic region and a hydrophobic region so that the biomaterials 120 may be provided in a limited region. More specifically, the plate member 110 may be coated with a hydrophilic material 140 and a hydrophobic material 150. As an example, as shown in FIG. 3, after one surface of the plate member 110 is coated with the hydrophobic material 150, only a portion thereof may be coated with the hydrophilic material 140. Alternatively, as shown in FIG. 4, one surface of the plate member 110 may be separately coated with the hydrophilic material 140 and the hydrophobic material 150.

The plate member 110 configured as described above may suppress the biomaterials 120 from being widely dispersed, since the biomaterials 120 are only provided in the portion thereof coated with the hydrophilic material 140.

A cell chip according to another embodiment of the present invention will be described with reference to FIGS. 5 and 6. For reference, in the present embodiment, components that are the same as those of the above-mentioned embodiment will be denoted by the same reference numerals and a detailed description thereof will be omitted.

The cell chip 100 according to the present embodiment of the invention is different from that according to the above-described embodiment in terms of the plate member 110.

The plate member 110 may have a plurality of protrusions 116. More specifically, the protrusions 116 may be formed at predetermined intervals on one surface of the plate member 110. For example, 532 (14×38) protrusions 116 may be formed on one surface of the plate member 110.

The biomaterials 120 may be restrictively provided on end surfaces of the protrusions 116. To this end, the end surfaces of the protrusions 116 may be coated with the hydrophilic material 140 as shown in FIG. 6. Meanwhile, although not shown in FIG. 6, circumferential surfaces of the protrusions 116 and remaining portions of the plate member 110 may be coated with a hydrophobic material.

In the cell chip 100 configured as described above, since the biomaterials 120 are only provided on the end surfaces of the protrusions 116, the biomaterials 120 may be easily cultured and attached using a well chip 400 (See FIG. 8). Meanwhile, in the case in which the drub is stored in the well chip 400, the reaction between the biomaterials 120 and the drug may be observed using the well chip 400.

Next, a method of manufacturing a cell chip will be described. For reference, in a description of the method of manufacturing a cell chip, the same components as those of the cell chip described above will be denoted by the same reference numerals.

A method of manufacturing a cell chip according to an embodiment of the present invention will be described with reference to FIG. 7.

The method of manufacturing a cell chip according to the present embodiment may include forming a hydrophobic film, forming a hydrophilic film, providing biomaterials, and forming a solidifiable layer.

### 1) Forming Hydrophobic Film

A hydrophobic film may be formed on the plate member 110. More specifically, one surface of the plate member 110 may be coated with the hydrophobic material 150 such that a thin film formed of the hydrophobic material 150 may be formed on one surface of the plate member 110. Here, the coating of the hydrophobic material 150 may be performed by a known method.

### 2) Forming Hydrophilic Film

A hydrophilic film may be formed on the plate member 110. More specifically, the hydrophobic film may be coated with the hydrophilic material 140. Here, the coating of the hydrophilic material 140 may only be performed on portions of the plate member on which the biomaterials 120 are to be provided. In addition, the hydrophilic material 140 may be generally coated in a circular shape.

### 3) Providing Biomaterials

The biomaterials 120 may be provided on the plate member 110. More specifically, the biomaterials 120 may be provided on the portions of the plate member coated with the hydrophilic material 140, that is, hydrophilic regions. Here, the biomaterials 120 may be provided using a known method.

### 4) Forming Solidifiable Layer

The solidifiable layer may be formed on the plate member 110. More specifically, the solidifiable material 130 may be applied to one surface of the plate member 110 to form the solidifiable layer having a predetermined height from one surface of the plate member 110. Here, the solidifiable layer may have a height enough to completely accommodate the biomaterials 120. In addition, as the solidifiable material 130, paraffin may be used.

A method of manufacturing a cell chip according to another embodiment of the present invention will be described with reference to FIG. 8.

The method of manufacturing a cell chip according to the present embodiment may include forming a hydrophilic film, providing biomaterials, pre-treating the biomaterials, and forming a solidifiable layer.

### 1) Forming Hydrophilic Film

A hydrophilic film may be formed on the plate member 110. More specifically, the protrusions of the plate member 110 may be coated with the hydrophilic material 140. More specifically, the ends of the protrusions 116 may be coated with the hydrophilic material 140. Meanwhile, although not shown in FIG. 8, the circumferential surfaces of the protrusions 116 may be additionally coated with the hydrophobic material as needed. For reference, the coating of the circumferential surfaces of the protrusions 116 with the hydrophobic material may be performed before the forming of the hydrophilic film.

### 2) Providing Biomaterials

The biomaterials 120 may be provided on the plate member 110. More specifically, the biomaterials 120 may be provided on the portions of the plate member coated with the hydrophilic material 140, that is, hydrophilic regions. Here, the biomaterials 120 may be provided using the well chip 400 as shown in FIG. 8. More specifically, the biomaterials 120 may be provided by immersing the protrusions 116 of the plate member 110 in the well chip 400 having the biomaterials 120 accommodated therein. Here, since the ends of the protrusions 116 are coated with the hydrophilic material 140, the biomaterials 120 may be easily attached to the ends of the protrusions 116. For reference, a reference numeral 410 of FIG. 8 may indicate grooves of the well chip 400 for accommodating the biomaterials 120 therein.

Meanwhile, although the case in which the biomaterials 120 are provided on the plate member 110 by a stamping process between the plate member 110 and the well chip 400 has been described in the present embodiment, a method of providing the biomaterials isnot limited to the stamping process. For example, the biomaterials may be provided on the plate member 110 in an injecting scheme using an inkjet print.

### 3) Pre-Treating Biomaterials

The biomaterials 120 may be allowed to react to drug. Alternatively, a chemical or physical action may be preliminarily applied to the biomaterials 120 so that the biomaterials 120 may easily react to the drug.

The pre-treating of the biomaterials 120 may be performed using the well chip 400. More specifically, the pre-treating of the biomaterials 120 may be performed by immersing the protrusions 116 of the plate member 110 in the well chip 400 having the drug 300 or a pre-treating material accommodated therein. Meanwhile, the pre-treating of the biomaterials 120 may also be performed by a method of injecting the drug or the pre-treating material without using the well chip 400.

### 4) Forming Solidifiable Layer

The solidifiable layer may be formed on the plate member 110. More specifically, the solidifiable material 130 may be applied to one surface of the plate member 110 to form the solidifiable layer having a predetermined height from one surface of the plate member 110. Here, the solidifiable layer may be formed to have a height enough to completely accommodate the biomaterials 120. In addition, as the solidifiable material 130, paraffin may be used.

The method of manufacturing a cell chip configured as described above may allow the biomaterials 120 to be easily provided on the protrusions 116.

Next, a cell slice sample according to an embodiment of the present invention will be described with reference to FIG. 9.

The cell slice sample 200 may include a slice 230 and biomaterials 220; 222, 224, 226, and 228.

The slice 230 may have a thin film shape. More specifically, the slice 230 may be obtained by cutting a solidified member having a predetermined height. The slice 230 may be formed of a solidifiable material. For example, the slice 230 may be formed of paraffin. However, the slice 230 is not limited to being formed of the paraffin, but may also be formed of any material capable of solidifying the biomaterials 220.

The biomaterials 220 may be provided on the slice 230. More specifically, the biomaterials 220 may be integrated with the slice 230 by the solidifiable material forming the slice 230.

Meanwhile, the biomaterials 220 may be formed of different tissues according to a position of the slice 230. For example, a first row among the biomaterials 220 provided on the slice 230 may be a first tissue 222, a second row thereamong may be a second tissue 224, a third row thereamong may be a third tissue 226, and a fourth row thereamong may be a fourth tissue 228. However, the biomaterials 220 provided on the slice 230 are not necessarily different tissues, but may be the same tissue as needed.

Since the cell slice sample 200 configured as described above includes the plurality of biomaterials 220 provided on the thin slice 230, it may facilitate drug experiment and inspection using the biomaterials 220. Further, in the cell slice sample 200, since a small amount of biomaterials 220 is provided integrally with the slice 230, a use amount of biomaterials 220 may be significantly decreased.

Next, a method of manufacturing a cell slice sample according to an embodiment and another embodiment of the present invention will be described with reference to FIGS. 10 and 11.

The method of manufacturing a cell slice sample may be based on the method of manufacturing a cell chip. For example, the method of manufacturing a cell slice sample according to the embodiment of the present invention may use the method of manufacturing a cell chip according to the embodiment of the present invention, and the method of manufacturing a cell slice sample according to another embodiment of the present invention may use the method of manufacturing a cell chip according to another embodiment of the present invention.

More specifically, the method of manufacturing a cell slice sample according to the embodiment of the present invention may include forming a hydrophobic film, forming a hydrophilic film, providing biomaterials, forming a solidifiable layer, and a cutting operation. Here, the forming of the hydrophobic film, the forming of the hydrophilic film, the providing of the biomaterials, and the forming of the solidifiable layer may be the same as or similar to those of the method of manufacturing a cell chip according to the embodiment of the present invention.

In addition, the method of manufacturing a cell slice sample according to another embodiment of the present invention may include forming a hydrophilic film, providing biomaterials, forming a solidifiable layer, and a cutting operation. Here, the forming of the hydrophilic film, the providing of the biomaterials, and the forming of the solidifiable layer may be the same as those of the method of manufacturing a cell chip according to another embodiment of the present invention.

That is, the method of manufacturing a cell slice sample may be different from the method of manufacturing a cell chip in that it further includes the cutting operation.

In the cutting operation the solidifiable layer may be cut in one direction (a direction parallel to one surface of the plate member). In addition, the cutting operation may include cutting the solidifiable layer to manufacture the cell slice sample 200. The cell slice sample 200 may have a thickness of 2 to 4 μm. Here, the cell slice sample 200 generated by the cutting operation may include various types of biomaterials 220. Therefore, drug experiments and inspections may be performed on the biomaterials 220 corresponding to the number of cell slice samples 200 obtained by cutting the solidifiable layer.

Meanwhile, since the cell slice sample 200 includes the plurality of biomaterials 220 in X axis and Y axis directions of the slice 200 (see FIG. 9), the cell slice sample 200 may be cut in the X or Y axis direction and be then used. In this case, the drug experiments and inspections using the sample slice sample 200 may be further performed.

As set forth above, according to embodiments of the present invention, since only a small amount of biomaterials may be used to perform a pre-treatment process such as culturing of the biomaterials, a drug treatment, and the like, and a slice of the pre-treated sample may be easily manufactured, time and costs required to analyze a reaction of the biomaterials may be decreased. In addition, according to the embodiments of the present invention, an experiment and an inspection of various types of biomaterials may be performed.

While the present invention has been shown and described in connection with the embodiments, it will be apparent to those skilled in the art that modifications and variations can be made without departing from the spirit and scope of the invention as defined by the appended claims.

What is claimed is:

1. A cell chip comprising:
a plate member;
biomaterials attached to one surface of the plate member; and
a solidifiable material formed on one surface of the plate member to form a layer including the biomaterials.

2. The cell chip of claim 1, wherein the plate member includes a hydrophilic region to which the biomaterials are attached and a hydrophobic region blocking the biomaterials from being attached thereto.

3. The cell chip of claim 2, wherein the hydrophobic region is formed to enclose the hydrophilic region.

4. The cell chip of claim 1, wherein the plate member includes protrusions to which the biomaterials are attached.

5. The cell chip of claim 4, wherein end surfaces of the protrusions are coated with a hydrophilic material so that the biomaterials are easily attached thereto.

6. The cell chip of claim 1, wherein the solidifiable material includes a paraffin component.

7. The cell chip of claim 1, wherein the biomaterials are sampled from at least one kind of sample.

8. A cell slice sample comprising:
a slice formed of a solidifiable material; and
at least one kind of biomaterials formed on the slice.

9. The cell slice sample of claim 8, wherein the slice is formed of a paraffin component.

10. The cell slice sample of claim 8, wherein the biomaterials are provided at predetermined intervals in first and second directions of the slice.

11. A method of manufacturing a cell chip, comprising:
attaching biomaterials to one surface of a plate member; and
forming a solidifiable layer on one surface of the plate member, the solidifiable layer accommodating the biomaterials therein and being formed of a solidifiable material.

12. The method of claim 11, further comprising:
applying a hydrophobic material to the plate member; and
partially applying a hydrophilic material to the hydrophobic material.

13. A method of manufacturing a cell chip, comprising:
attaching biomaterials to protrusions formed on one surface of a plate member; and
forming a solidifiable layer on one surface of the plate member, the solidifiable layer accommodating the biomaterials therein and being formed of a solidifiable material.

14. The method of claim 13, further comprising:
applying a hydrophobic material to the plate member; and
partially applying a hydrophilic material to the hydrophobic material.

15. The method of claim 13, wherein the attaching of the biomaterials includes immersing the protrusions of the plate member in grooves in which the biomaterials are accommodated.

16. A method of manufacturing a cell slice sample, comprising:
attaching biomaterials to one surface of a plate member;
forming a solidifiable layer on one surface of the plate member, the solidifiable layer accommodating the biomaterials therein and being formed of a solidifiable material; and
cutting the solidifiable layer into a slice.

17. The method of claim 16, further comprising:
applying a hydrophobic material to the plate member; and
partially applying a hydrophilic material to the hydrophobic material.

18. A method of manufacturing a cell slice sample, comprising:
attaching biomaterials to protrusions formed on one surface of a plate member;
forming a solidifiable layer on one surface of the plate member, the solidifiable layer accommodating the biomaterials therein and being formed of a solidifiable material; and
cutting the solidifiable layer into a slice.

19. The method of claim 18, further comprising applying a hydrophobic material to the protrusions.
